(19) United States
(12) Patent Application Publication
(10) Pub. No.: US 2019/0216835 A1
(43) Pub. Date: Jul. 18, 2019

(54) Inhibition of Zika Virus Infection

(71) Applicant: University of Massachusetts, Boston, MA (US)

(72) Inventor: Abraham L. Brass, Newton, MA (US)

(21) Appl. No.: 16/306,842

(22) PCT Filed: Jun. 6, 2017
(86) PCT No.: PCT/US17/36053

§ 371 Date: Dec. 3, 2018

Related U.S. Application Data
(60) Provisional application No. 62/348,039, filed on Jun. 9, 2016.

(51) Int. Cl. A61K 31/7048; A61K 31/7056; A61K 31/35; A61P 31/14

(52) U.S. Cl. CPC A61K 31/7048; A61P 31/14; A61K 31/35; A61K 31/7056

(57) ABSTRACT

Methods for treating or reducing the risk of developing Zika virus infection in a subject, comprising administering an effective amount of lanatoside C, ribavirin and/or ivermectin to the subject.

### CLAIM OF PRIORITY

This application claims the benefit of U.S. Provisional Patent Application Ser. No. 62/348,039, filed on Jun. 9, 2016. The entire contents of the foregoing are hereby incorporated by reference.

### TECHNICAL FIELD

Described herein are methods for treating or reducing the risk of developing Zika virus infection in a subject, comprising administering an effective amount of ribavirin, ivermectin, and/or lanatoside C to the subject.

### BACKGROUND

Zika virus disease, caused by infection with the Zika virus, is an emerging global health threat that can cause severe birth defects including microcephaly and other neurological disorders. On Feb. 1, 2016, the World Health Organization (WHO) declared Zika virus a Public Health Emergency of International Concern (PHEIC). There is presently no approved therapy for Zika virus.

### SUMMARY

The present invention is based, at least in part, on the discovery of small molecules that are capable if inhibiting replication of the Zika virus.

Thus, provided herein are methods for treating or reducing the risk of developing Zika virus infection in a subject. The methods include administering an effective amount of lanatoside C, ribavirin and/or ivermectin to the subject.

In some embodiments, the subject has been exposed to the Zika virus, or lives or is planning to visit an area in which the Zika virus is endemic.

In some embodiments, the subject has been diagnosed with the Zika virus.

In some embodiments, the subject is an adult male or female, e.g., an adult male or female who is sexually active.

In some embodiments, the subject is a pregnant woman. In some embodiments, the subject is a pregnant woman, and the method comprises intravenous administration of ribavirin, ivermectin, and/or lanatoside C. In some embodiments, the lanatoside C is deslanatoside.

Also provided herein is the use of lanatoside C, ribavirin and/or ivermectin in treating or reducing the risk of developing Zika virus infection in a subject. In some embodiments, the subject has been exposed to the Zika virus, or lives or is planning to visit an area in which the Zika virus is endemic. In some embodiments, the subject has been diagnosed with the Zika virus. In some embodiments, the subject is an adult male or female, e.g., an adult male or female who is sexually active. In some embodiments, the subject is a pregnant woman. In some embodiments, the lanatoside C, ribavirin and/or ivermectin is formulated for intravenous administration, e.g., wherein the subject is a pregnant woman.

In some embodiments, the lanatoside C is deslanatoside.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a bar graph showing the results of experiments wherein Vero cells were infected for 48 h with Zika virus MR766 and stained for DNA and double stranded viral RNA. The indicated compounds at the indicated concentrations were added at the time of infection. Mean percent infected cells of n=3 experiments are shown +/−SD.

FIG. 2 is a set of images of Vero cells infected for 48 h with Zika virus MR766 and stained for DNA (blue) and double stranded viral RNA (green). Either Lanatoside 6 nM or DMSO (v/v) was added at the time of infection. Mean percent infected cells of n=3 experiments are shown +/−SD.

### DETAILED DESCRIPTION

The new millenium has brought a rapid expansion of human flavivirus infections, including dengue viruses (DENV), yellow fever virus (YFV), West Nile virus (WNV) and Zika virus (ZIKV) (Bhatt et al., 2013). Given that global warming is predicted to expand the range of the insect vectors which carry these viruses, it is critical that we understand their biology so as to design effective therapies. DENV and ZIKV are single-stranded positive-sense RNA viruses that are transmitted to humans by Aedes mosquitos. Both are rapidly expanding health threats producing an escalating number of infections in the Americas and worldwide. Each year, 390 million people are infected with DENV, with 500,000 individuals hospitalized with severe dengue, the majority of those being young children (Bhatt et al., 2013). ZIKV, first isolated from an infected macaque in Uganda in 1947, suddenly emerged in Micronesia in 2007 and expanded its range to Southeast Asia. In May 2015, ZIKV was identified in Brazil coincident with an upsurge in neurologic and fetal abnormalities. With its rapid spread to Central and South America, ZIKV has emerged as a severe health threat by virtue of its fast paced global spread and its associated morbidities, including microcephaly and Guillain-Barre syndrome. (D'Ortenzio et al., 2016) (Driggers et al., 2016; Haug et al., 2016; Lazear and Diamond, 2016; Musso and Gubler, 2016) (Rasmussen et al., 2016). These events have led to ZIKV being declared a public health emergency by the WHO. Recent animal models have demonstrated that ZIKV infects the placentas of pregnant mice with transmission to fetal mice resulting in death or severe growth impairment (Lazear et al., 2016; Miner and Diamond, 2016; Miner et al., 2016; Li et al., 2016). There are no specific therapies for flavivirus infection, although a DENV vaccine has recently been approved in some countries. There is no approved vaccine or therapy for ZIKV infection.

Flavivirus replication begins with the virus binding to host cell receptors and undergoing endocytosis (Fernandez-Garcia et al., 2009). A number of proteins have been implicated as facilitating DENV attachment and entry, including TIM1 and AXL (Jemielity et al., 2013; Meertens et al., 2012; Morizono and Chen, 2014; Perera-Lecoin et al., 2014; Richard et al., 2015), the latter having also been identified as an important ZIKV entry factor (Hamel et al., 2015). Subsequent to intial viral entry, late endosomal acidification triggers the fusion of host and viral membranes and permits the virus' positive sense RNA genome (vRNA) to enter the host cell cytosol. Upon cytosolic entry, the vRNA is translated into a large polyprotein on the rough endoplasmic reticulum (RER). This polyprotein is processed by both host and viral proteases into three structural proteins (premembrane (prM), capsid (C) and the glycoprotein envelope (E protein)), and seven non-structural (NS) proteins (NS1, NS2A, NS2B, NS3, NS4A, NS4B, NS5). DENV has been demonstrated to extensively remodel the ER into replication centers (RCs), where progeny viruses are created. The newly synthesized flaviviruses then traffic from the RER to the cell surface via the Golgi, where they undergo exocytosis, thus spreading the infection to neighboring cells.

The flaviviruses have a complex lifecycle which relies on the host cell's proteins, pathways and other resources. Earlier efforts have addressed the role of arthropod DENV-host factors (Sessions et al., 2009) and human factors required by the related flaviviruses, YFV (Le Sommer et al., 2012) and WNV (Krishnan et al., 2008; Ma et al., 2015). Nonetheless, fundamental questions regarding how human proteins modulate flavivirus replication, including ZIKV infection, remain.

Methods of Treatment

The methods described herein include methods for the treatment or reduction of risk of infection with Zika virus, e.g., of Zika virus disease. Generally, the methods include administering a therapeutically effective amount of ribavirin, ivermectin, and/or lanatoside C or a related cardiac glycoside as described herein, to a subject who is in need of, or who has been determined to be in need of, such treatment.

In the present methods the treatment can be administered, e.g., to a subject who has been exposed to the Zika virus, or who lives or is planning to visit an area in which the Zika virus is endemic, or who has been diagnosed with the Zika virus. In some embodiments, the subject is a pregnant woman; optionally, these methods can include intravenous administration of ribavirin, ivermectin, and/or lanatoside C. In some embodiments, the subject is an adult male or female, e.g., an adult male or female who is sexually active, e.g., who lives or is planning to visit an area in which the Zika virus is endemic. In some embodiments, the subject is an infant or a child, e.g., a newborn (0-3 months), infant (3 months to 1 year), toddler (2-4 years), child (5-12 years), or adolescent (13-18 years).

Lanatoside C

Lanatoside C (3β-[4-O-β-D-Glycopyranosyl-4-O-(3-O-acetyl-β-D-digitoxopyranosyl)-4-O-β-D-digitoxopyranosyl-β-D-digitoxopyranosyl]-12β,14-dihydroxy-5β,14β-card-20(22)-enolid) is a cardiac glycoside obtained from the leaf of Digitalis lanata that is believed to acts by inhibiting the Na+-K+-ATPase pump. It is US Food and Drug Administration (FDA)-approved for the treatment of congestive heart failure and cardiac arrhythmia, and has recently been shown to inhibit some negative-strand RNA viruses including Herpes Simplex Virus and Influenza Virus (Dodson et al. 2007; Hoffmann et al., 2008; Shi et al., 2016) and positive sense ssRNA viruses including Kunjin Virus (flavivirus), Chikungunya virus (alphavirus), SINV (alphavirus), human enterovirus 71, and Dengue Virus (flavivirus) (Cheung et al., 2014).

Although the present methods exemplify the use of Lanatoside C, other related molecules can also be used, e.g., digoxin, oleandrin, acetyldigoxin, digitoxin, k-strophanthin beta, gitoxin, gitoxigenin, periplocymarin, strophantine octahydrate, convallatoxin, digoxigenin, helveticoside, digitoxigenin (e.g., digitoxigenin acetate), peruvoside, acocantherine, cymarin, strophanthidin acetate, strophantine octahydrate, sarmentogenin, ouabain, sarmentoside B, nerifolin, deslanoside, or proscillaridin.

Lanatoside C can be administered intravenously or orally; when administered intravenously deslanoside (desacetly-lanoside C) can be used.

Ribavirin

Ribavirin (1-[(2R,3R,4S,5R)-3,4-dihydroxy-5-(hydroxymethyl)oxolan-2-yl]-1H-1,2,4-triazole-3-carboxamide), a nucleoside inhibitor, is a guanosine (ribonucleic) analog used to stop viral RNA synthesis and viral mRNA capping. It is presently used for, e.g., treating RSV and hepatitis C infections.

Ivermectin

Ivermectin (22,23-dihydroavermectin B1a+22,23-dihydroavermectin B1b) is an anti-parasitic in the avermectin family; its mechanism of action is increasing cell membrane permeability, resulting in paralysis and death of the parasite.

Pharmaceutical Compositions and Methods of Administration

The methods described herein include the use of pharmaceutical compositions comprising lanatoside C, ribavirin, and/or ivermectin as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, and oral administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Dosage

The present methods include administration of an effective amount of ribavirin, ivermectin, and/or lanatoside C. An “effective amount” is an amount sufficient to effect beneficial or desired results. For example, a therapeutic amount is one that achieves the desired therapeutic effect (e.g., reduction in viral titer). This amount can be the same or different from a prophylactically effective amount, which is an amount necessary to prevent onset of disease or disease symptoms (e.g., reduction in risk of infection). An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day, e.g., for one week, two weeks, three weeks, four weeks, one month, two months, three months, four months, five months, six months, or more. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. In some embodiments, the methods can include orally administering an initial (loading) dose of 1.0-2 mg with a maintenance dose of 0.25-1 mg/day for adults, and in children a loading dose of 0.02-0.05 mg/lb of body weight/day for children with maintenance dose of about 100-200 μg/day. For intravenous administration, the dosing of deslanoside can be 0.8-1.2 mg initially followed by 0.4 mg doses every 2-4 hours as needed in adults, or 0.01 mg/lb bodyweight/day in children. In some embodiments, the methods include orally administering doses below those provided above, e.g., a dose that does not have effects on cardiac function, e.g., a daily oral dose of 0.1-0.2 mg/day for adults or a dose of less than 0.01 mg/lb bodyweight/day for children.

### EXAMPLES

The invention is further described in the following examples, which do not limit the scope of the invention described in the claims.

### Example 1. Inhibition of Zika Virus

The effects of a number of small molecules previously reported to be effective in inhibiting replication of related viruses were evaluated on replication of the Zika virus using an immunofluorescence readout for viral protein expression. Molecules tested included Ivermectin 18898-1G; Mycophenolic acid M3536-50MG; Brequinar SML0113-5MG; Sodium Oxamate O2751-5G; Chloroquine C6628-25G; Hydroxychloroquine H0915-5MG; and Lanatoside C L2261-100MG.

Materials and Methods

Small molecules: The following compound were all purchased from Sigma: Ivermectin 18898-1G, Mycophenolic acid 3536-50MG, Brequinar SML0113-5MG, Sodium Oxamate O2751-5G, Chloroquine C6628-25G, Lanatoside C L2261-100MG, Ribavirin R9644-10MG, and resuspended in DMSO.

Cells: Vero cells (ATCC, CCL-81) were cultured in complete Dulbecco's Modified Eagle Media (Sigma) with 10% FBS (Invitrogen) and 2 mM L-glutamine (Invitrogen).

Viruses: Zika virus strain MR766 was kindly provided by Dr. Robert Tesh at the World Reference Center for Emerging Viruses and Arboviruses at the University of Texas Medical Branch in Galveston Tex. MR766 was obtained originally from a Rhesus macaque in Uganda in 1947. Viruses were propagated in Vero cells (ATCC) (Dick et al., 1952) and the titer determined by standard plaque assays and immunofluorescence imaging assays for E protein expression.

Infection assays: Vero cells were plated the night before in a 384-well plate format. The following day the cells were infected with ZIKV MR766 at an MOI of 0.3-0.5 in the presence of the indicated compounds or the DMSO control (v/v). 48 hr post-infection the cells are fixed with formalin, permeabilized with 0.1% Triton-X100 and immunostained using the 4G2 monoclonal antibody against the E protein. The cells were then incubated with an Alexa Fluor 488 goat anti-mouse secondary and stained for DNA with Hoechst 33342. The cells were imaged on an automated Image Express Micro (IXM) microscope at 4× magnification. Images were analyzed using the MetaXpress software program to determine the total cells per well, and the percentage of infected cells in each well.

Results

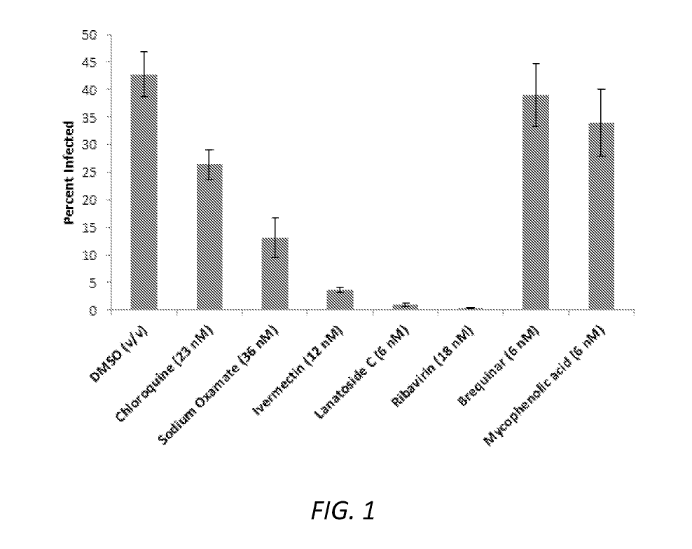

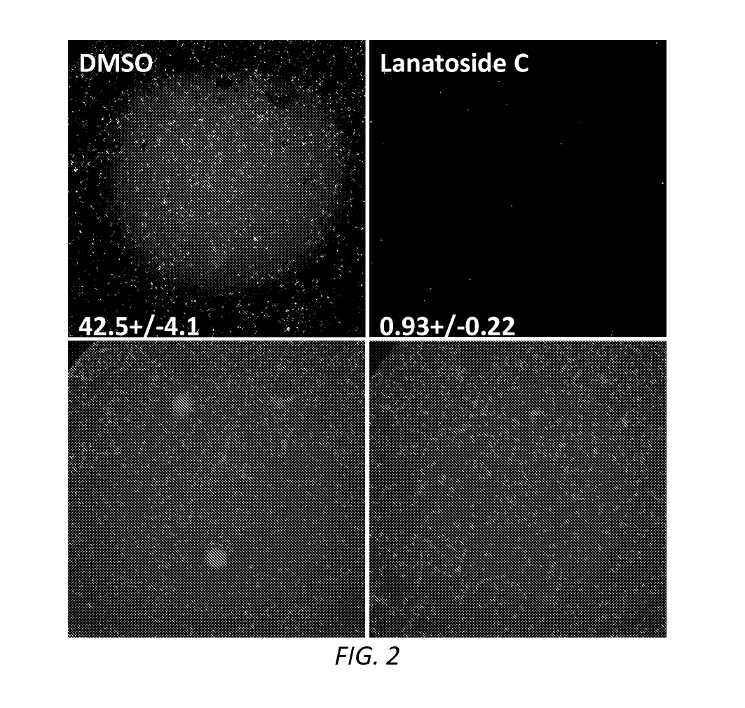

Although all of the small molecules tested had been shown to be effective in stopping replication of related viruses including other flaviviruses, surprisingly, only three of the small molecules tested (ribavirin, ivermectin and Lanatoside C) had any significant effect in the present assay (FIG. 1). Lanatoside C was effective at stopping replication of the virus, as shown in FIG. 2.

### REFERENCES

Bhatt et al. (2013). The global distribution and burden of dengue. Nature 496, 504-507.
Cheung et al. (2014) Antiviral activity of lanatoside C against dengue virus infection. Antiviral Res. 2014 November; 111:93-9.
D'Ortenzio et al. (2016). Evidence of Sexual Transmission of Zika Virus. The New England journal of medicine. N Engl J Med. 2016 Jun. 2; 374(22):2195-8
Dodson et al. (2007) Inhibitors of the sodium potassium ATPase that impair herpes simplex virus replication identified via a chemical screening approach. Virology. 366, 340-348.
Driggers et al. (2016). Zika Virus Infection with Prolonged Maternal Viremia and Fetal Brain Abnormalities. N Engl J Med. 2016 Jun. 2; 374(22):2142-51
Fernandez-Garcia et al. (2009). Pathogenesis of flavivirus infections: using and abusing the host cell. Cell host & microbe 5, 318-328.
Hamel et al. (2015). Biology of Zika Virus Infection in Human Skin Cells. Journal of virology 89, 8880-8896.
Haug et al. (2016). The Zika Challenge. The New England journal of medicine. 374:1801-1803
Hoffmann et al. (2008) Modulation of influenza virus replication by alteration of sodium ion transport and protein kinase C activity. Antiviral Res. 80, 124-134.
Jemielity et al. (2013). TIM-family proteins promote infection of multiple enveloped viruses through virion-associated phosphatidylserine. PLoS pathogens 9, e1003232.
Krishnan et al. (2008). RNA interference screen for human genes associated with West Nile virus infection. Nature 455, 242-245.
Lazear and Diamond. (2016). Zika Virus: New Clinical Syndromes and its Emergence in the Western Hemisphere. Journal of virology. J Virol. 2016 Apr. 29; 90(10):4864-75
Lazear et al. (2016). A Mouse Model of Zika Virus Pathogenesis. Cell host & microbe. Cell Host Microbe. 2016 May 11; 19(5):720-30
Le Sommer et al. (2012). G protein-coupled receptor kinase 2 promotes flaviviridae entry and replication. PLoS neglected tropical diseases 6, e1820.
Li et al. (2016). Zika Virus Disrupts Neural Progenitor Development and Leads to Microcephaly in Mice, Cell Stem Cell. pii: S1934-5909(16)30084-4.
Ma et al. (2015). A CRISPR-Based Screen Identifies Genes Essential for West-Nile-Virus-Induced Cell Death. Cell reports 12, 673-683.
Meertens et al. (2012). The TIM and TAM families of phosphatidylserine receptors mediate dengue virus entry. Cell host & microbe 12, 544-557.
Miner and Diamond. (2016). Understanding How Zika Virus Enters and Infects Neural Target Cells. Cell stem cell 18, 559-560.
Miner et al. (2016). Zika Virus Infection during Pregnancy in Mice Causes Placental Damage and Fetal Demise. Cell. 165(5):1081-91.
Morizono and Chen. (2014). Role of phosphatidylserine receptors in enveloped virus infection. Journal of virology 88, 4275-4290.
Musso and Gubler. (2016). Zika Virus. Clinical microbiology reviews 29, 487-524.
Perera-Lecoin et al. (2014). Flavivirus entry receptors: an update. Viruses 6, 69-88.
Rasmussen et al. (2016). Zika Virus and Birth Defects—Reviewing the Evidence for Causality. N Engl J Med. 2016 May 19; 374(20):1981-7
Richard et al. (2015). Virion-associated phosphatidylethanolamine promotes TIM1-mediated infection by Ebola, dengue, and West Nile viruses. Proceedings of the National Academy of Sciences of the United States of America 112, 14682-14687.
Richard et al. (2013). Biosynthesis of ionotropic acetylcholine receptors requires the evolutionarily conserved ER membrane complex. Proceedings of the National Academy of Sciences of the United States of America 110, E1055-1063.
Sessions et al. (2009). Discovery of insect and human dengue virus host factors. Nature 458, 1047-1050.
Shi et al. (2016) Lanatoside C Promotes Foam Cell Formation and Atherosclerosis. Sci Rep. 6:20154.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or reducing the risk of developing Zika virus infection in a subject, the method comprising identifying a subject who has or is at risk of developing a Zika virus infection, and administering an effective amount of one or more of lanatoside C, ribavirin or ivermectin to the subject.

2. The method of claim 1, wherein the subject has been exposed to the Zika virus, or lives or is planning to visit an area in which the Zika virus is endemic.

3. The method of claim 1, wherein the subject has been diagnosed with the Zika virus.

4. The method of claim 1, wherein the subject is an adult male or female.

5. The method of claim 1, wherein the subject is a pregnant woman.

6. The method of claim 3, wherein the subject is a pregnant woman, and the method comprises intravenous administration of ribavirin, ivermectin, and/or lanatoside C.

7. (canceled)

8. (canceled)

9. (canceled)

10. (canceled)

11. (canceled)

12. (canceled)

13. The method of claim 6, wherein the lanatoside C is deslanatoside.

14. (canceled)

15. The method of claim 4, wherein the subject is an adult male or female who is sexually active.
